(19) United States
(12) Patent Application Publication
(10) Pub. No.: US 2026/0016483 A1
(43) Pub. Date: Jan. 15, 2026

(54) HIGH-THROUGHPUT BIOPRODUCT IDENTIFICATION

(71) Applicant: UCHICAGO ARGONNE, LLC, Chicago, IL (US)

(72) Inventors: Gyorgy Babnigg, Countryside, IL (US); Jessica L. Johnson, Plainfield, IL (US); Philip D. Laible, Downers Grove, IL (US); Rosemarie Wilton, Elmhurst, IL (US); Chase Akins Akins, Marengo, IL (US)

(21) Appl. No.: 19/204,892

(22) Filed: May 12, 2025

Related U.S. Application Data
(60) Provisional application No. 63/670,565, filed on Jul. 12, 2024.

(51) Int. Cl. G01N 33/68; C12Q 1/24

(52) U.S. Cl. CPC G01N 33/6848; C12Q 1/24

(57) ABSTRACT

A method for identifying cells producing desired biological products may include: creating, from a suspension of candidate cells in medium, a plurality of droplets, each droplet comprising either a single cell from the suspension of candidate cells or cell-free medium; incubating the plurality of droplets; breaking the plurality of droplets to release surviving cells; retrieving the surviving cells; growing colonies of the surviving cells; and testing the colonies of the surviving cells, also called candidate cells, to determine an amount of a desired product produced. Determining how much of the desired product is produced by the candidate cells may involve high-throughput mass-spectroscopy techniques, and in some aspects, may be made at multiple time points to establish the best performing cell lines over time.

### CROSS REFERENCE TO RELATED APPLICATION

The benefit of priority to U.S. Provisional Application No. 63/670,565 filed Jul. 12, 2024, is hereby claimed and the disclosure is incorporated herein by reference in its entirety.

### STATEMENT REGARDING FEDERALLY SPONSORED RESEARCH OR DEVELOPMENT

This invention was made with government support under DE-AC02-06CH1137 awarded by the Department of Energy. The government has certain rights in this invention.

### FIELD OF THE DISCLOSURE

The present disclosure relates to methods and systems for the identification of cells strains which rapidly multiply, thrive, and exhibit tolerance in selected environments. The methods and systems find particular use in research settings where the initial candidates for viable cells multiply poorly in a selected medium.

### BACKGROUND

Identification of cell lines capable of thriving in a given environment (e.g., medium and temperature range) is traditionally accomplished by beginning with a large volume of medium in which cells are suspended. The suspension with a mixture of cells is divided into flasks, vials, or wells of cell culture plates. After incubation and reaching saturation, cultures aliquots are diluted and transferred into new culture vessels (passaging). The procedure is repeated for months to produce evolved candidates, due to spontaneous mutations or epigenetic changes, with rapid multiplication in the given environment. In the end, the cultures are spread onto agar plates, colonies picked, isolates analyzed characterized, and top performers selected, those that both thrive in a given environment and produce a desired biological product. The characterization can be carried out using traditional analytical methods (e.g., HPLC, LC-MS) or using biosensors with either a fluorescent protein (FP) or enzyme reporter. Such traditional methods to date take months to evolve the hardiest cells. This is possibly due to the fact that poor performers can feed on byproducts produced by the top performers (cross-feeding).

The results from cells that are tested which produces a desired bioproduct gives results for the milieux or mixture of cells. There may a suppression or amplification effect from a particular group of cells which are cultured together. This may not allow for the best producers to be identified, in turn possibly preventing optimization of the production of targeted bioproducts.

In addition to the time needed for spontaneous mutation using conventional adaptive laboratory evolution (ALE), the conventional analysis methods require much time due to sample preparation (e.g., filtering) or due to instrument constraints. Further, conventional instruments used to date for characterization of the growth and product, or analyte, production of cell strains are not able to process large numbers of samples. Because of this, cell growth and analyte production cannot be simultaneously monitored over time with sampling intervals less than hours.

### SUMMARY OF THE DISCLOSURE

The disclosure thus provides a method for determining an amount of a product or analyte produced by cells in a select environment, the method comprising steps including: creating a plurality of droplets, wherein each droplet comprises a single cell in a medium; collecting the plurality of droplets; incubating the plurality of droplets to allow cell replication; breaking the plurality of droplets to release surviving cells and the medium; and measuring the amount of the product or analyte produced by the cells using a high-throughput mass spectrometry system (HTP-MS system). In some aspects, the method further comprises after breaking the plurality of droplets to release surviving cells, collecting the surviving cells; re-encapsulating the surviving cells in a further plurality of droplets; and incubating the plurality of droplets for at least 2 days before retrieving the surviving cells. In some aspects, the method further comprises creating colonies from cell isolates from the surviving cells; and using the (HTP-MS) system to determine the amount of the product or analyte present in an aliquot of supernatant from each cell isolate. In some aspects, the method further comprises removing an aliquot of supernatant periodically from each culture isolate and comparing the amount of desired product in the aliquot corresponding to each culture isolate with that of control cells to produce a plot or table of the amount of desired product for each culture isolate as a function of time. In some aspects of the method, testing the colonies of the surviving cells comprises utilizing UV ionization on each aliquot. In some aspects, the select environment comprises any one or more of: percent oxygen (O₂), relative humidity, temperature, culture medium composition, pH, and agitation. In some aspects, the culture medium composition comprises any of: a feed-stock; a toxin; an inhibitor; a salt; ionic strength; high/low osmolarity; a biological product or byproduct; a media component; and cell debris/waste. In some aspects, each droplet of the plurality of droplets has a volume of no more than 1 nanoliter. In some aspects, each droplet of the plurality of droplets has a diameter of not more than 100 microns. In some aspects, the method further comprises repeating steps comprising: creating a plurality of droplets; collecting the plurality of droplets; incubating the plurality of droplets to allow cell growth; and breaking the plurality of droplets to release surviving cells; at least 2 times.

The disclosure also provides a system comprising: a droplet producing apparatus configured to accept cells in a medium and create a plurality of droplets, each droplet comprising a single cell; an incubator configured to allow replication of cells inside each droplet; a droplet breaking apparatus adapted to accept the plurality of droplets from the incubator and yield cells in suspension; a cell culture apparatus adapted to accept cells in suspension from the droplet breaking apparatus, the cell culture apparatus configured to allow for creation of supernatant for testing for cell isolates; a liquid handling system adapted to create an array of aliquots of supernatant, wherein each aliquot corresponds to each cell isolate; and a high-throughput mass spectroscopy (HTP-MS) instrument, the high-throughput mass spectroscopy instrument configured to test each aliquot. In some aspects of the system, the HTP-MS instrument comprises an ultraviolet (UV) ionization component. In some aspects, the HTP-MS instrument is adapted to determine an amount of a desired product or analyte produced by each cell isolate as a function of time. In some aspects, the droplet producing apparatus is further configured to accept the cells in suspension created by the droplet breaking apparatus. In some aspects, the system further comprises a cell dilution apparatus adapted to accept the cells in suspension created by the droplet breaking apparatus, wherein the cell dilution apparatus is configured to provide the droplet forming apparatus with cells in the medium. In some aspects, the droplets are created using a surfactant in a fluorinated oil, further wherein the droplet breaking apparatus comprises a liquid capable of extracting and repartitioning the surfactant. In some aspects, the system is configured to repeat, until a predetermined number of repetitions is achieved, the steps of: incubating the cells for a predetermined time period; accepting, by a cell dilution apparatus, cells in suspension from the droplet breaking apparatus; and providing, to the droplet producing apparatus, cells in the medium from the cell dilution apparatus. In some aspects, the predetermined time period is at least 12 hours. In some aspects, the predetermined number of repetitions is at least 2 times, at least 3 times, at least 4 times, at least 5 times, at least 6 times, at least 7 times, at least 8 times, at least 9 times, at least 10 times. In some aspects, the steps further comprise creating cultures in a multi-well plate from the cells in suspension received from the droplet breaking apparatus.

The disclosure herein also provides a method of determining an amount of a product produced by candidate cells in culture using the system described hereinabove, the method comprising: performing a dALE (droplet adaptive laboratory evolution) process, the process comprising: creating, using the droplet producing apparatus, a plurality of droplets from an initial suspension of cells; incubating the plurality of droplets in a select environment using the incubator to yield an incubated plurality of droplets; providing the incubated plurality of droplets to the droplet breaking apparatus; breaking the plurality of droplets using the droplet breaking apparatus to yield cells in suspension; and re-encapsulating, by the droplet producing apparatus, the cells in suspension provided by the droplet breaking apparatus; and after multiple iterations performing the dALE process, measuring the amount of the product or analyte produced by the cells and a rate of replication of the cells.

### BRIEF DESCRIPTION OF THE DRAWINGS

The figures described below depict various aspects of the apparatus, systems, and methods disclosed therein. It should be understood that each figure depicts an example of a particular aspect of the disclosed apparatus, systems, and methods, and that each of the figures is intended to accord with a possible example thereof. Further, wherever possible, the following description refers to the reference numerals included in the following figures, in which features depicted in multiple figures are designated with consistent reference numerals.

There are shown in the drawing arrangements which are discussed herein, it being understood, however, that the examples of the disclosure are not limited to the precise arrangements and instrumentalities shown, wherein:

FIG. 1 is a schematic of a system for identification of cells which produce desired biological products.

FIG. 2 is a schematic of a system for encapsulation of a cell in a droplet of medium.

FIG. 3A-3C shows Pseudomonas cell growth in droplets over time. FIG. 3A shows a representative droplet containing a single cell in a certain medium, at an initial time shortly after formation of the droplet. FIG. 3B shows a representative droplet after one day of incubation. FIG. 3C shows a representative droplet after two days of incubation.

FIG. 4 shows a process using the system of FIG. 1.

FIG. 5 provides a schematic of steps of a method for determining cells which thrive in a given medium to produce a desired product using the system of FIG. 1; and

FIGS. 6A and 6B show sample data obtained utilizing a system according to FIG. 1 as detailed in the description of example 1.

### DETAILED DESCRIPTION

The disclosure is directed to efficient, low volume methods and systems for identifying cells which can flourish in various environments while producing desired bioproducts. Such methods and systems utilize droplet adaptive laboratory evolution (dALE) and a rapid molecular species identification technique utilizing high-throughput mass spectroscopy (HTP-MS). Identification of desirable cells using these method and system is accomplished in a matter of days or weeks, as opposed to the weeks or months required by conventional methods.

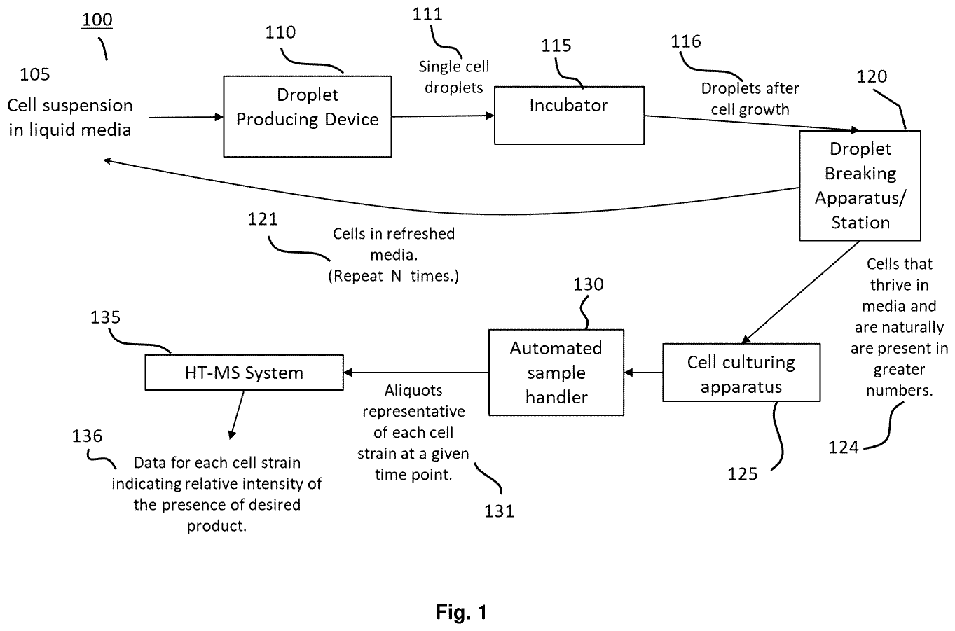

Turning to the Figures, FIG. 1 shows a system 100 for identifying cells which produce desired biological products. In various aspects, the system 100 includes a cell suspension 105 (i.e., cell solution), a droplet producing device 110, an incubator, a droplet breaking 120 apparatus or station, a cell culturing 125 apparatus or station, an automated sample handling apparatus 130, and a high-throughput mass spec apparatus 135.

Within the system, the types of products or information passed between components of the system 100 are shown as well. Types of products and information passed include any combination of: single cell droplets 111, droplets after cell growth 116, cells that thrive in the selected medium 124, cells in refreshed medium 121, supernatant aliquots representative of each cell at a given time point 131, and data for each cell indicating the relative intensity of the presence of a desired product 136.

The portions of the system that are used in dALE are those for creating droplets, incubation, droplet breaking, and introduction of fresh or refreshed medium when preparing for another iteration of the dALE process. Droplet adaptive laboratory evolution (dALE), is useful in that it is used in the methods described herein to find the cell lines which not only thrive in a given medium, but that also optimally produce a desired biological product while using the principle of survival of the fittest. ALE (adaptive laboratory evolution) determines similar information, but it uses a mixture of candidate cell lines in medium-filled beakers or wells. In contrast, dALE isolates cells in such a way that removes any effects that one cell would have on another as they grow together. Further, the decreased medium volume when using dALE versus ALE is a resources saving.

Returning to the figures, the cell suspension 105 represented in FIG. 1 includes cells that are believed to be suited to survival in a chosen, or select environment. The initial suspension of cells includes any of: a genetically heterogenous or inhomogeneous cell population, a genetically homogenous cell population, natural cells, engineered cells, and a heterogeneous cell population generated by library approaches (e.g., transposon library, CRISPR-Cas9 library, cells expressing a library of enzyme or pathway variants in the engineered host). The environment includes variables external to the droplets and those internal to the droplets. Environmental variables external to the droplets include any one or more of: percent oxygen (O₂), relative humidity, temperature, and agitation. Variables internal to the droplets include the growth medium, or culture medium, used in the suspension. The selected growth medium, in some aspects, includes any of culture medium, minimal medium, selective medium, differential medium, transport medium, and indicator medium. In various aspects, the growth medium is of a specific pH or ionic strength (i.e., salt content) which is challenging for certain cells. Alternatively, or additionally, the growth medium provides a specific type of nutrient, or feed-stock, e.g., starch, glucose. Other characteristics that may vary in the medium include an amount of, or presence of: a toxin, cell debris, a biological product, waste, an inhibitor, and any combination thereof. In some aspects, the cells present in the cell suspension 105 include preselected cells that are known to produce a desired product in the selected growth medium. This cell suspension 105 of preselected cells in the selected medium is provided to the droplet producing device 110. In some aspects, the cells are selected to produce a desired product, and dALE process allows for selection of cells that are hardy and able to rapidly multiply in a medium that includes the desired product and impurities associated with the desired product. Exposure to the first and subsequent mediums results in a small subset of cells adapt to the various mediums and growth conditions in some aspects due to accumulation of spontaneous mutations with positive effects.

The system and method include a droplet producing device 110. In some aspects, the droplet producing device 110 includes an active device, a passive device, traditional methods, ultrasonic droplet formation, atomization, a microfluidic chip-based method, off-chip methods, a process the proceeds from off-chip to on-chip then to off-chip, and a two-stream flow-focusing microfluidic device that accepts cells in the cell suspension 105.

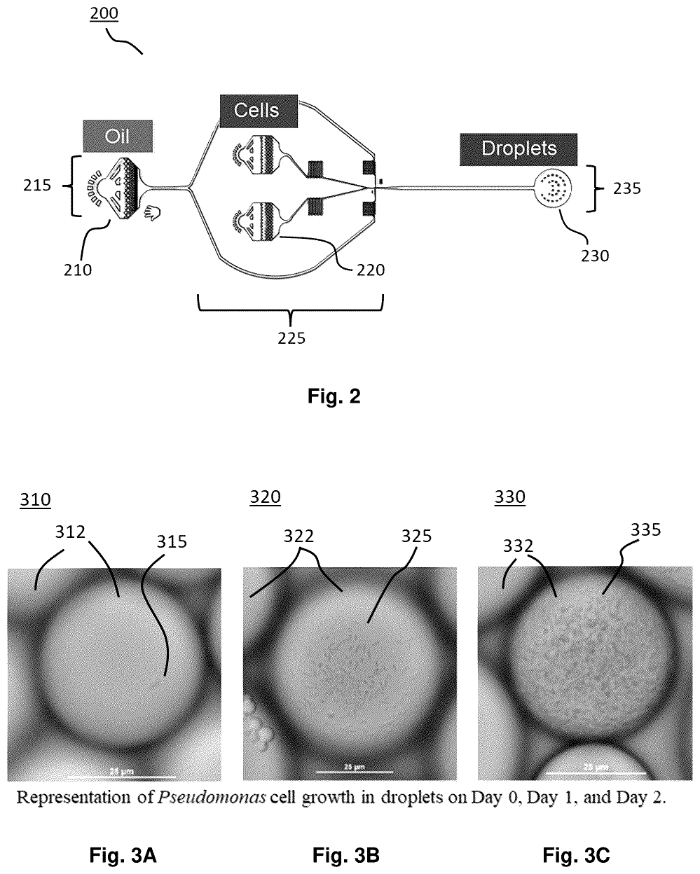

When the droplet producing device is a two-stream flow-focusing microfluidic device 110, the device includes a chip. In some implementations, the chip is a cell encapsulation PDMS chip 200, as shown in FIG. 2. The PDMS cell encapsulation chip 200 includes, or is fluidly connected to, a reservoir of oil 210 and a feature 215 on the chip 200 for directing oil flow on the chip 200. Further, in some examples, the chip 200 includes a cell suspension reservoir 220 which feeds into a portion 225 of the chip 200 which draws up cells from the reservoir 220 and dilutes cells surrounded by the selected medium in oil from the oil reservoir 210. The configuration of the feature 225 of the chip 200 allows for the creation of droplets with a distribution of the number of encapsulated cells, with the majority of the distribution of encapsulated cells leads to droplets containing no more than one cell each. These droplets are captured at another location 235 on the chip 200 and collected in a collection reservoir or repository 230 before being placed in an incubator (115 in FIG. 1). Other configurations are possible for the cell encapsulation chip 200 as well as for the droplet producing device 110.

In such a droplet producing device 110 as shown in FIG. 2, droplets are formed from the cell suspension 105 by dilution of the cell solution with a fluorinated oil. In such aspects, the droplets are created using a surfactant in the fluorinated oil, such as perfluoropolyether (PFPE).

Droplets produced by the droplet producing device 110 range in size from about 5 μm (microns) to about 2000 μm, about 10 μm to about 1000 μm, about 15 μm to about 500 μm, about 20 μm to about 100 μm, 30 μm to about 80 μm, such as from about 35 μm to about 75 μm, including from about 40 μm to about 70 μm, such as from about 45 μm to about 65 μm, and including from about 50 μm to about 60 μm. In some implementations, the droplets range in size from about 10 μm to about 150 μm in diameter, such as from about 15 μm to about 125 μm, including from about 20 μm to about 100 μm. Each droplet produced by the droplet producing device 110 has a volume less than 1 microliter, such as less than 1 nanoliter, including less than 900 picoliters, such as less than 900 picoliters, such as less than 800 picoliters, less than 700 picoliters, less than 650 picoliters, less than 600 picoliters, less than 550 picoliters, less than 500 picoliters, less than 450 picoliters, less than 400 picoliters, less than 350 picoliters, less than 300 picoliters, less than 250 picoliters, less than 200 picoliters, less than 150 picoliters, less than 100 picoliters, less than 90 picoliters, less than 80 picoliters, less than 70 picoliters, less than 60 picoliters, including less than 50 picoliters. In some implementations, each droplet has a volume of less than 45 picoliters. In various aspects, each droplet produced by the droplet producing device 110 has a volume of about 1 picoliters to about 1000 nanoliters, such as about 1 picoliter to about 900 nanoliters, about 2 picoliters to about 800 picoliters, about 3 picoliters to about 700 picoliters, about 4 picoliters to about 600 picoliters, about 5 picoliters to about 500 picoliters, such as about 25 microliters to about 35 microliters.

The droplet producing device 110 has a rate of encapsulation at least at 1000 droplets per second, capable of the production ranging from 1.0 million droplets to about 3 million droplets, such as from about 1.25 million to about 2.75 million droplets, including from about 1.5 million to about 2.5 million droplets from a predetermined amount of cell suspension 105 in less than an hour. In some aspects, the droplet producing device 110 has a rate of encapsulation of about 2.3 million droplets from about 200 μl (microliters) of cell suspension 105 in less than an hour.

Referring back to FIG. 1, the droplet producing device 110 passes droplets with either no cells (i.e., only the selected medium, cell-free medium) or a single cell 111 to an incubator 115 or incubation station. The incubator 115 allows the cells to grow within the droplets over time. Incubation involves maintaining the collection of droplets in oil at the optimal temperature for the given organism (normally between 25 deg. C. and 38 deg. C.). Droplets are stable between 10 deg. C. to about 90 deg. C., when dALE is used to adapt cells to higher temperatures or mezzo-or-thermophiles are the host cells. dALE can be performed in either aerobic or anerobic conditions. In some aspects, the droplets in oil are gently agitated so as to provide constant aeration, or gas exchange, of the droplets. The extra fluorinated oil amended with the surfactant minimizes the oil-droplet emulsion from drying out. Those cells which are better suited to growing in the selected environment produce more progeny cells over time within the droplet. FIGS. 3A-3C show the increase in the number of cells in a representative droplet over time. FIG. 3A shows an initial condition 310, in which a droplet 312 with a single cell 315 is shown surrounded by other droplets 312. In some aspects, the initial condition 310 is the environment on the day that the droplet was formed, as well as when the medium within the droplet is fresh, and free from the products created by the cell. FIG. 3B shows a later condition 320. In the case shown in FIG. 3A-3C, the later condition 320 shown is after a day (i.e., 24 hours) has passed. The aging droplet 322 has an increased number of cells 325. FIG. 3C shows a droplet condition 330 at day two (i.e., 48 hours since starting the study). In FIG. 3C, the further aged droplets 332 are shown. One of the droplets 332 has a great number of cell 335 within the aged cell 332 in the center. For example, FIG. 3A-3C show the progressive increase in Pseudomonas cells in a predetermined environment over time.

In the dALE process, after incubating the cells, the droplets are broken at a droplet breaking apparatus or station 120, and the contents of the droplets are suspended into medium 121 and are exposed to an environment. The environment is the same as the initial environment, in some implementations. Alternatively, in some implementations, the environment into which the cells resulting from broken droplets are exposed is different from the initial environment. The initial environment and the subsequent environments may be the same or different. In some aspects, the initial environment and the subsequent environments differ in one or more constituents or characteristics. The subsequent cell suspension 105 is again presented to the droplet producing device 110, incubator 115 (as droplets), and the droplet breaking apparatus/station 120. This cycle, i.e., the incubating, breaking, and re-encapsulating steps is repeated multiples times, such that enrichment of the cells in suspension is observed. The number of repetitions of this cycle includes at least 2 times, at least 3 times, at least 4 times, at least 5 times, at least 6 times, at least 7 times, at least 8 times, at least 9 times, at least or about 10 times, at least or about 20 times, at least or about 30 times, at least or about 40 times, at least or about 50 times, at least or about 60 times, at least or about 70 times, at least or about 80 times, at least or about 90 times, and at least or about 100 times or more. In some aspects, the method for generating and/or selecting cells adapted to replicate in a select environment, i.e., the dALE process is repeated about 4 to 10 times, such as about 5 to 9 times, including about 6 to 8 times in some implementations. In some aspects, the incubating, breaking, and re-encapsulating steps are repeated at least about 10 times over a period of about one to two months. The number of repetitions may be a predetermined number or it may be based on a sampling of droplets and the growth of cells within the droplet samples.

The droplets are separated from the oil following the iterations or repetitions of the growth and suspension in new media. In some implementations, the separation of the droplets and surrounding oil is accomplished using a membrane such as a fine mesh stainless steel membrane. Alternatively, in some implementations, an emulsion breaking solution is added to the droplets, resulting in an aqueous and oil phase. The cell suspension may then be recovered from the aqueous phase.

The droplet breaking apparatus or station 120 includes one or more of any suitable device, apparatus, or assembly which removes the oil, or other liquid used to create the droplets, from the environs of the droplets. In some aspects, the droplet breaking device of apparatus includes a liquid capable of extracting and repartitioning a surfactant used in droplet formation. The product of the droplet breaking apparatus 120 includes candidate cells representative of candidate cells. These cells are prepared and mixed with new medium.

Following a number of repetitions of the droplet formation and incubation process (dALE), the cells, which are no longer in droplets, are cultured in growth medium. The growth medium is a suitable medium including growth medium in one or more well plates. This occurs after cells 124 that thrive in the selected environment used in droplet formation and incubation are passed to a cell culturing apparatus 125.

The cells which are cultured represent cells which were able to grow in the select environment. In the dALE process, as individual cells are encapsulated with each iteration of the droplet formation and incubation process, each cell that is able to grow in significant numbers in the selected environment may be present, with those better suited to growth in the selected environment being present in greater numbers. The dALE process favors the proliferation of cells which are able to quickly reproduce and thrive in the select environment, as the period between cell encapsulation and droplet breaking is relatively short, such as less than 10 days, less than 9 days, less than 8 days, including less than 6 days. In some implementations, the period of time between cell encapsulation and droplet breaking in the dALE process is between 12 hours and 5 days, including between 1 day and 4 days, such as between 2 and 3 days.

In an example of how dALE (droplet adaptive laboratory evolution) will come to select the cells that are best suited to thriving in a select environment, a cell of strain X may grow well resulting in 200 cells in a droplet after a first iteration of the droplet and incubation process. These 200 cells are then encapsulated into their own droplets, one cell per droplet, at the start of the second iteration. At the end of the second iteration, cell X will be represented by at least 40000 cells in the suspension. Then, at the end of the third iteration, assuming that each of the 40000 cells, each in its own droplet, divide resulting in 200 cells per droplet, there will be 8 million cells of strain X resulting from the initial droplet. If strain Y grows slower and resulting in 50 cells per droplet for each iteration, beginning with one cell of strain Y in a single droplet, after three iterations, strain Y will be represented 125000 cells. Then, at the end of three iterations of dALE cell cultivation, the number of cells of strain X will outnumber those of strain Y by 64:1. Most importantly, a cell strain Z may not be able to grow at all in the given condition, resulting in 3 cells at the end of round 3 and therefore practically eliminated from the population.

The dALE process allows for selection of rapidly replicating, and potentially rapidly evolving, cells which are best suited to a select environment. Combining the ability to quantify the rate of replication with the ability to determine the amount of production of an analyte or product over time allows cell strains which still increase analyte production over time though cell proliferation has reached a plateau. This knowledge can be useful in subsequent experiments once promising cell strains have been identified. To this end, an analysis system which combines the ability to determine cell density, and in turn cell reproduction, with the ability to determine a relative quantity of an analyte (e.g., a bioproduct) is described herein. The HTP-MS methods and systems described herein are utilized after a process (e.g., conventional adaptive laboratory evolution (ALE), dALE) has identified the cells that are best suited to a select environment to characterize the cell replication and analyte (e.g., bioproduct) production of those identified cells. These HTP-MS methods and systems are compatible with any protocol yielding a large number of isolates that need characterization in parallel using a fast method.

As shown in FIG. 1, the system 100 includes an automated sample handler 130 which creates an array of aliquots in sample wells on one or more plates. Each well on the one or more plates represents a cell which showed desirable growth after culturing by the cell culturing apparatus 125. In some aspects, the cell culturing apparatus 125 includes a neutral growth medium into which the isolates derived from the broken droplets are cultivated. As indicated above, the growth medium is any of: basal medium, agar, and/or broth. The cell culturing apparatus 125 includes any of: culture plates, test tubes, and/or well plates.

The automated sample handler 130 extracts an aliquot of liquid 131 containing products of the cells from each well. The cell cultures are spun down to separate solids (e.g., cell debris) form supernatant from which aliquots are extracted. These aliquots are diluted to form representative samples. The dilution is greater than 1:10, such as 1:20, 1:30, 1:40, 1:50, 1:60, including greater than 1:90, such as greater than 1:100. The representative samples are then passed to the high-throughput mass spec (HTP-MS) system 135 without any preprocessing, such that the representative sample are directly injected into the HTP-MS system. The HTP-MS system 135 collects data 136 corresponding to each cell. The data is indicative of the presence of the desired product. Assuming the automated sample handler 130 extracts representative samples from the supernatant aliquots corresponding to each cell in a well of a plate that are of uniform or standardized volume and the number of isolated cells in each well is also standardized or uniform, the data 136 may also represent the relative ability of each cell to produce the desired product. The HTP-MS system 135 is calibrated at the beginning and end of each run by running a known sample through the system.

The high-throughput mass spec (HTP-MS) system 135 includes an ionizing component or array (i.e., ionizer) and a mass spec array or instrument. The HTP-MS system 135 is able to provide data 136 for each cell without any preprocessing of the contents of aliquots extracted by the automated sample handler 130. This is done by employing ionization apparatus and techniques which have the ability to narrow the range of mass to charge (m/z) ratios examined by the mass spec portion of the HTP-MS system 135. Knowledge of the m/z ratios indicative or characteristic of the desired product is needed when selecting a portion of the mass spec data to focus on for determination of the best performing cells.

Example ionizers used to directly produce ions without preprocessing include any of: UV ionizers, x-ray ionizers, and ambient ionization apparatus. For UV and x-ray ionizers, selecting or tuning the energy of the ionizing photons (e.g., the wavelength of UV light) may be used to produce a photoionization efficiency curve. The photoionization efficiency curve, in conjunction with the m/z ratio may provide a fingerprint for a desired product (i.e., molecules or ions formed during the ionization process with the desired product as the source). Comparing or tracking peaks in the photoionization efficiency curve for particular m/z ratios may indicate the better performing cells.

In some aspects, other mass spec techniques are used to determine ionic species, and in turn the desired products, without the use of liquid chromatography or other separation or digestive techniques. These mass spec techniques include any of: time-of-flight (TOF) mass spec, quadrupole analyzer mass spec, and/or a quadrupole ion traps.

Data from the HTP-MS is tracked over time for each of the candidate cells, such as cells resulting from the dALE process. In some aspects, this tracking involves repeated extraction of aliquots 131 by the automated sample handler 130 at given time intervals or at predetermined time points. This type of data may indicate an inflection point in the amount of desired product produced by a cell versus time. The inflection point may be a factor in selection of preferred candidate cells for further experimentation or examination.

Turning once again to FIG. 1, once data from the HTP-MS system is used to determine the cell proliferation and analyte production the preferred candidate cells (e.g., cells identified or resulting from experimentation or a process such as dALE), further testing of the preferred candidate cells is conducted outside of the system 100 shown in FIG. 1.

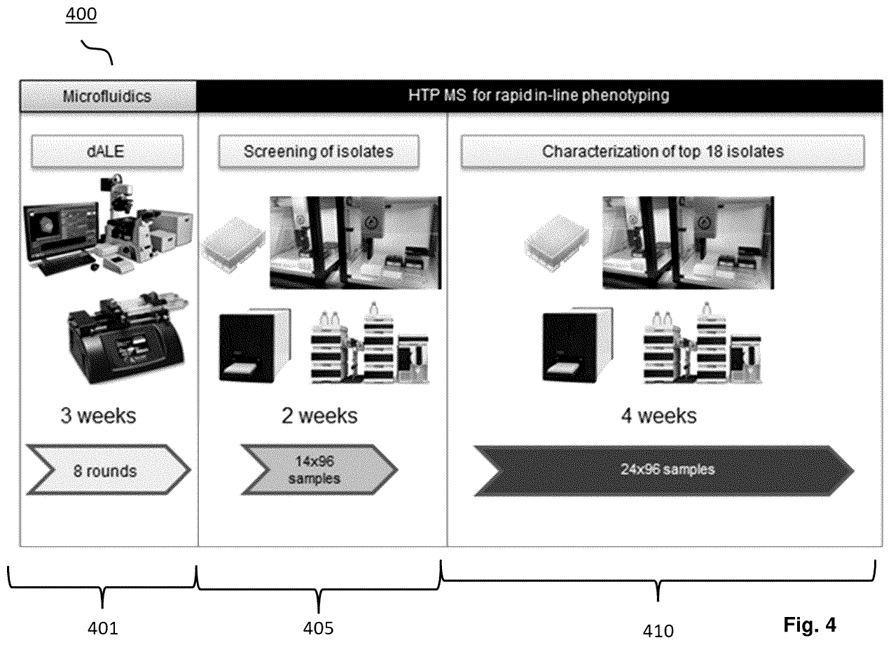

The timing of the process supported by the system 100 shown in FIG. 1 is one of the advantages of the system 100 as compared to conventional means of identifying preferred candidate cells. FIG. 4 illustrates the possible time frames 400 for various steps in the process utilizing the system 100. In some aspects, the microfluidics stage 401, has a time frame of around 3 weeks for 8 iterations, or rounds, of the dALE process, which is the droplet formation and incubation process. Other time frames for the dALE process include any of: around 2 weeks for 5 iterations, around 3 weeks for 8 iterations, around 4 weeks for 11 iterations, around 5 weeks for 13 iterations, and/or around 7 weeks for 19 iterations. In some implementation, multiple rounds of the dALE process are performed for at least 2 weeks, including for at least 3 weeks, at least 4 weeks, at least 5 weeks, at least 10 weeks, at least 20 weeks, at least 50 weeks, and including at least 100 weeks. The screening process 405 has a time frame of 2 weeks for about 1344 samples (e.g., 14 well plates of 96 wells each) in some aspects. Time frames for the screening process 405 include any of: around 1 week for about 1344 samples, around 2 weeks for about 1344 samples, and/or 3 weeks for about 1920 samples.

The screening process 405 utilizes the cell culturing apparatus 125 and HTP-MS system 135. Following the screening process 405, the identification or determination of the top candidates 410 requires 4 weeks for 2304 samples (e.g., 24 plates of 96 wells each), in some aspects. In such aspects, the identification process 410 employs the HTP-MS system 135, as described above. The screening 405 and identification 410 processes are part of a large process of high-throughput pass spec rapid in-line phenotyping.

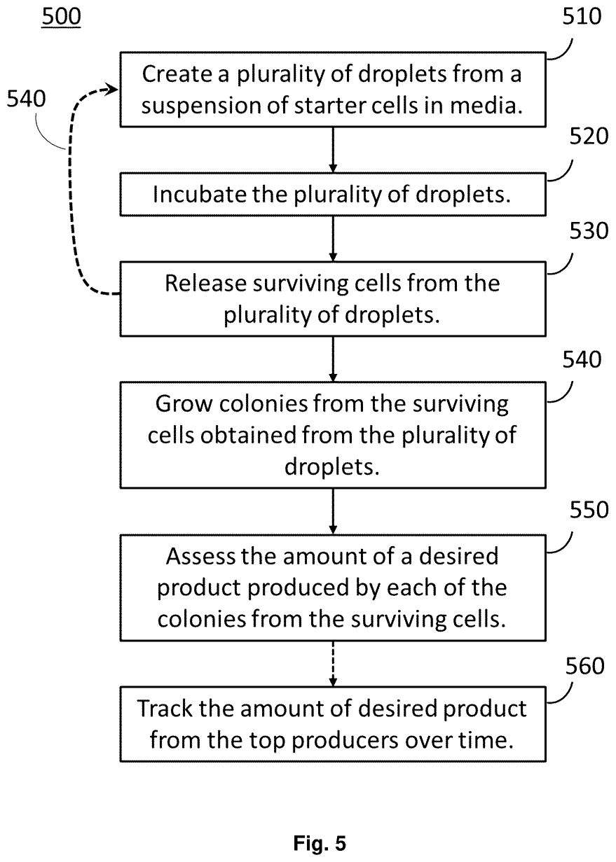

FIG. 5 shows an example method 500 for use of the systems and processes described above. In the method 500, a plurality of droplets are formed from a suspension of a range of cells as in step 510. As discussed above, the medium used in the suspension may be antagonistic for the cell type used. The droplets are then incubated as in step 520 in a predetermined environment that includes the medium. Then the droplets are broken and the surviving cells are released from the plurality of droplets as in step 530. These steps 510, 520, 530 may be repeated multiple times as in step 540. After multiple iterations, or repeats of steps 510, 520, and 530, the surviving cells are released from the final plurality of droplets (step 530), and the method involves growing colonies from the surviving cells as in step 540. Then the HTP-MS system 135 of the system 100 assesses a replication rate and the amount of the desired product produced by each cell obtained from the earlier steps, as shown in step 550.

### Example 1

In an example, four engineered cells of Pseudomonas putida, GB062, LC040, LC071, and LC238 were evolved in M9-30 mM DMR-Carbenicillin.

### Sampling and Cell Replication Monitoring Protocol:

A 96 deep-well plate is set up with 1.25 mL media with the isolates diluted to a starting 0.1 OD600 value. The plate is sealed and placed into an incubator shaker.

A first sample (t0) is obtained for OD600 characterization immediately after a brief shaking.

250 μL (microliter) of culture are withdrawn into a 96-well plate, the deep-well plate is sealed and placed back into the incubator immediately after the culture withdrawal. The 96-well plate with the aliquot is read on the plate reader for OD600 absorbance. The plate is centrifuged at 2700×g (5 min) to settle the bacteria. 20 μL of the supernatant is transferred into a separate 96-well plate that has 180 μL 0.5% formic acid in every well prior to the addition of supernatant. The content of each of the wells is mixed with the pipette after the addition of the 20 μL aliquot and transferred to the HTP-MS measurement. The operation is automated via two liquid handlers.

During subsequent sampling, 20 μL culture is transferred into a 96-well containing 180 μL water in each well for cell dilution. The contents are mixed and OD600 measured with a plate reader (acquiring cell density, proliferation data) and subsequently centrifuged (2700×g for 5 min). As with sample preparation for the previous iteration of HTP-MS, a 20 μL of the supernatant is transferred into a separate 96-well plate that has 180 μL 0.5% formic acid in each well prior to the addition of the supernatant. The content is mixed with the transferring pipette after the addition of the 20 μL aliquot. This mixture is transferred to the HTP-MS measurement.

### Mass Spectrometry Protocol:

The HTP-MS instrument is configured such that an autosampler is directly connected to an ionizing component, and the samples are then passed to the mass spectrometer. Software can be used to control the pumps, autosampler, detector, and mass spectrometer associated with the instrument.

The flow rate of the mobile phase (0.5% formic acid in water) is set at 0.4 mL/min and a baseline is established for the ionizing component and the mass spectrometer.

The mass spectrometer is set to monitor 4 distinct m/z values (e.g., 141 m/z for monitoring cis,cis-muconate bioproduct). Potential m/z values can be between 80-1500 m/z using the current instrument.

Once a stable baseline is established, 2 μL samples are injected one minute apart. A standard curve is established before each run to calibrate samples across the entire time-course.

The combination of automated sample preparation and HTP-MS approach enables the generation of analytical readouts within 2 hours when one 96-well plate is used for culturing, enabling culture sampling every 2 hours.

### Data:

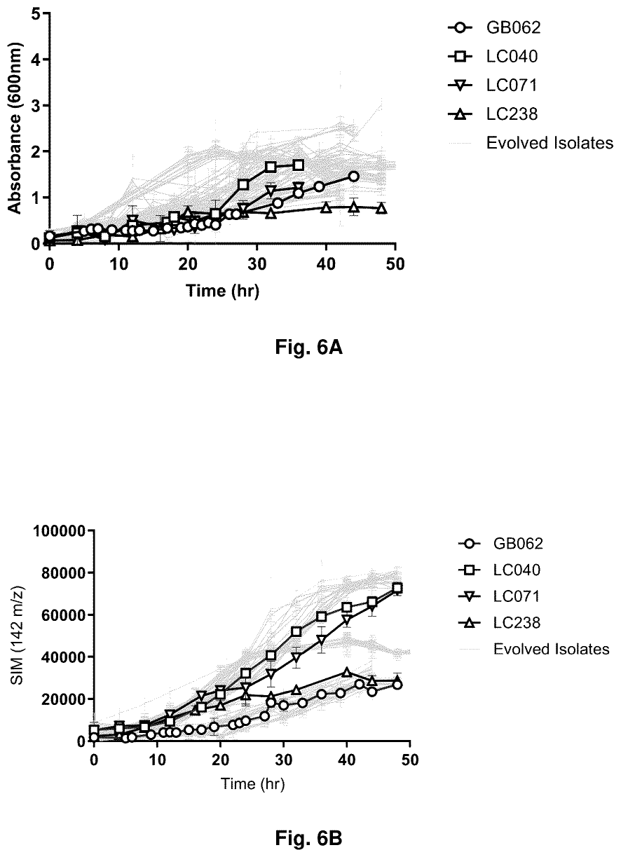

The plot shown in FIG. 6A shows the absorbance of samples from those cell lines at various time points. The absorbance units correlate to optical density which in turn correlates to the number of bacteria in a given sample. The plot shown in FIG. 6B illustrates the relative intensity or counts of ionized particles for each of these strains as a function of time. The counts are made for a m/z (mass to charge ratio) of 142. Comparison of the plots in FIGS. 6A and 6B illustrates that slowing multiplication or replication of a cell strain over time does not necessarily correlate to slowing analyte production.

The disclosure provides a method for determining an amount of a desired product produced by candidate cells in culture, comprising creating a plurality of droplets, wherein each droplet comprises either cell-free medium or a single cell in medium; collecting the plurality of droplets; creating from a suspension of candidate cells in medium a plurality of droplets, each droplet comprising either cell-free medium, or a single cell from the suspension of candidate cells; incubating the plurality of droplets to allow cell growth; breaking the plurality of droplets to release surviving cells; retrieving the surviving cells; growing colonies of the surviving cells; and measuring the amount of a desired product produced by the surviving cells.

Though the HTP-MS systems and methods described herein are discussed largely with respect to use following the dALE process, these systems and methods are compatible with any process which is used to determine or create cells that are adapted to thrive in a select environment. The HTP-MS systems and methods are applicable to processes such as ALE as well as dALE. Additionally, or alternately, the HTP-MS systems and methods described herein can be used with cells such as engineered cells and a heterogeneous cell population generated by library approaches, as well as with any cells chosen for analysis.

The following additional considerations apply to the foregoing discussion. Throughout this specification, plural instances may implement functions, components, operations, or structures described as a single instance. Although individual functions and instructions of one or more methods are illustrated and described as separate operations, one or more of the individual operations may be performed concurrently, and nothing requires that the operations be performed in the order illustrated. Structures and functionality presented as separate components in exemplary configurations may be implemented as a combined structure or component. Similarly, structures and functionality presented as a single component may be implemented as separate components. These and other variations, modifications, additions, and improvements fall within the scope of the subject matter herein.

As used herein any reference to “some examples” or “one example” or “an example” means that a particular element, feature, structure, or characteristic described in connection with the example is included in at least one example. The appearances of the phrase “in one example” in various places in the specification are not necessarily all referring to the same example.

Some examples may be described using the expression “coupled” and “connected” along with their derivatives. For example, some examples may be described using the term “coupled” to indicate that two or more elements are in direct physical or electrical contact. The term “coupled,” however, may also mean that two or more elements are not in direct contact with each other, but yet still cooperate or interact with each other. The examples are not limited in this context.

As used herein, the terms “comprises,” “comprising,” “includes,” “including,” “has,” “having” or any other variation thereof, are intended to cover a non-exclusive inclusion. For example, a function, process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the examples herein. This is done merely for convenience and to give a general sense of the description. This description should be read to include one or at least one and the singular also includes the plural unless it is obvious that it is meant otherwise.

Upon reading this disclosure, those of skill in the art will appreciate still additional alternative structural and functional designs for methods and systems for producing a reusable structural member through the disclosed principles herein. Thus, while particular examples and applications have been illustrated and described, it is to be understood that the disclosed examples are not limited to the precise construction and components disclosed herein. Various modifications, changes, and variations, which will be apparent to those skilled in the art, may be made in the arrangement, operation and details of the method and apparatus disclosed herein without departing from the spirit and scope defined in the appended claims.

The following list of aspects reflects a variety of the examples explicitly contemplated by the disclosure. Those of ordinary skill in the art will readily appreciate that the aspects below are neither limiting of the examples disclosed herein, nor exhaustive of all the examples conceivable from the disclosure above but are instead meant to be exemplary in nature.

1. A method for determining an amount of a product or analyte produced by cells in a select environment, the method comprising steps including:
creating a plurality of droplets, wherein each droplet comprises a single cell in a medium;
collecting the plurality of droplets;
incubating the plurality of droplets to allow cell replication;
breaking the plurality of droplets to release surviving cells and the medium; and
measuring the amount of the product or analyte produced by the cells using a high-throughput mass spectrometry system (HTP-MS system).

2. The method of aspect 1, further comprising:
after breaking the plurality of droplets to release surviving cells, collecting the surviving cells;
re-encapsulating the surviving cells in a further plurality of droplets; and
incubating the plurality of droplets for at least 2 days before retrieving the surviving cells.

3. The method of aspect 1 or 2, further comprising:
creating colonies from cell isolates from the surviving cells; and
using the (HTP-MS) system to determine the amount of the product or analyte present in an aliquot of supernatant from each cell isolate.

4. The method of any one of aspects 1-3, wherein the method further comprises removing an aliquot of supernatant periodically from each culture isolate and comparing the amount of desired product in the aliquot corresponding to each culture isolate with that of control cells to produce a plot or table of the amount of desired product for each culture isolate as a function of time.

5. The method of aspect 4, wherein testing colonies of the surviving cells comprises utilizing UV ionization on each aliquot.

6. The method of any one of aspects 1-5, wherein the select environment comprises any one or more of: percent oxygen (O₂), relative humidity, temperature, culture medium composition, pH, and agitation.

7. The method of aspect 6, wherein the culture medium composition comprises any of:
a feed-stock;
a toxin;
an inhibitor;
a salt;
ionic strength;
high/low osmolarity;
a biological product or byproduct;
a media component; and
cell debris/waste.

8. The method of any one of aspects 1-7, wherein each droplet of the plurality of droplets has a volume of no more than 1 nanoliter.

9. The method of any one of aspects 1-8, wherein each droplet of the plurality of droplets has a diameter of not more than 100 microns.

10. The method of aspect 1, further comprising repeating the steps of creating a plurality of droplets;
collecting the plurality of droplets;
incubating the plurality of droplets to allow cell growth; and
breaking the plurality of droplets to release surviving cells;
at least 2 times.

11. A system comprising:
a droplet producing apparatus configured to accept cells in a medium and create a plurality of droplets, each droplet comprising a single cell;
an incubator configured to allow replication of cells inside each droplet;
a droplet breaking apparatus adapted to accept the plurality of droplets from the incubator and yield cells in suspension;
a cell culture apparatus adapted to accept cells in suspension from the droplet breaking apparatus, the cell culture apparatus configured to allow for creation of supernatant for testing for cell isolates;
a liquid handling system adapted to create an array of aliquots of supernatant, wherein each aliquot corresponds to each cell isolate; and
a high-throughput mass spectroscopy (HTP-MS) instrument, the high-throughput mass spectroscopy instrument configured to test each aliquot.

12. The system of aspect 11, wherein the HTP-MS instrument comprises an ultraviolet (UV) ionization component.

13. The system of any of aspects 11-12, wherein the HTP-MS instrument is adapted to determine an amount of a desired product or analyte produced by each cell isolate as a function of time.

14. The system of any of aspects 11-13, wherein the droplet producing apparatus is further configured to accept the cells in suspension created by the droplet breaking apparatus.

15. The system of any of aspects 11-14, further comprising a cell dilution apparatus adapted to accept the cells in suspension created by the droplet breaking apparatus, wherein the cell dilution apparatus is configured to provide the droplet producing apparatus with cells in the medium.

16. The system of any of aspects 11-15, wherein the droplets are created using a surfactant in a fluorinated oil, further wherein the droplet breaking apparatus comprises a liquid capable of extracting and repartitioning the surfactant.

17. The system of any of aspects 11-16, wherein the system is configured to repeat, until a predetermined number of repetitions is achieved, steps comprising:
incubating the cells for a predetermined time period;
accepting, by a cell dilution apparatus, cells in suspension from the droplet breaking apparatus; and
providing, to the droplet producing apparatus, cells in the medium from the cell dilution apparatus.

18. The system of aspect 17, wherein the predetermined time period is at least 12 hours.

19. The system of aspect 17 or 18, wherein the predetermined number of repetitions is at least 2 times, at least 3 times, at least 4 times, at least 5 times, at least 6 times, at least 7 times, at least 8 times, at least 9 times, at least 10 times.

20. The system of any one of aspects 17-19, wherein the steps further comprise creating cultures in a multi-well plate from the cells in suspension received from the droplet breaking apparatus.

21. A method of determining an amount of a product produced by candidate cells in culture using the system of aspect 11, the method comprising:
performing a dALE process, the process comprising:
creating, using the droplet producing apparatus, a plurality of droplets from an initial suspension of cells;
incubating the plurality of droplets in a select environment using the incubator to yield an incubated plurality of droplets;
providing the incubated plurality of droplets to the droplet breaking apparatus;
breaking the plurality of droplets using the droplet breaking apparatus to yield cells in suspension; and
re-encapsulating, by the droplet producing apparatus, the cells in suspension provided by the droplet breaking apparatus; and
after multiple iterations performing the dALE process, measuring the amount of the product or analyte produced by the cells and a rate of replication of the cells.

What is claimed is:

1. A method for determining an amount of a product or analyte produced by cells in a select environment, the method comprising steps including:
creating a plurality of droplets, wherein each droplet comprises a single cell in a medium;
collecting the plurality of droplets;
incubating the plurality of droplets to allow cell replication;
breaking the plurality of droplets to release surviving cells and the medium; and
measuring the amount of the product or analyte produced by the cells using a high-throughput mass spectrometry system (HTP-MS system).

2. The method of claim 1, further comprising:
after breaking the plurality of droplets to release surviving cells, collecting the surviving cells;
re-encapsulating the surviving cells in a further plurality of droplets; and
incubating the plurality of droplets for at least 2 days before retrieving the surviving cells.

3. The method of claim 1, further comprising:
creating colonies from cell isolates from the surviving cells; and
using the (HTP-MS) system to determine the amount of the product or analyte present in an aliquot of supernatant from each cell isolate.

4. The method of claim 1, wherein the method further comprises removing an aliquot of supernatant periodically from each culture isolate and comparing the amount of desired product in the aliquot corresponding to each culture isolate with that of control cells to produce a plot or table of the amount of desired product for each culture isolate as a function of time.

5. The method of claim 4, wherein testing colonies of the surviving cells comprises utilizing UV ionization on each aliquot.

6. The method of claim 1, wherein the select environment comprises any one or more of: percent oxygen (O₂), relative humidity, temperature, culture medium composition, pH, and agitation.

7. The method of claim 6, wherein culture medium composition comprises any of:
a feed-stock;
a toxin;
an inhibitor;
a salt;
ionic strength;
high/low osmolarity;
a biological product or byproduct;
a media component; and
cell debris/waste.

8. The method of claim 1, wherein each droplet of the plurality of droplets has a volume of no more than 1 nanoliter and/or wherein each droplet of the plurality of droplets has a diameter of not more than 100 microns.

9. The method of claim 1, further comprising repeating the steps of creating a plurality of droplets;
collecting the plurality of droplets;
incubating the plurality of droplets to allow cell growth; and
breaking the plurality of droplets to release surviving cells;
at least 2 times.

10. A system comprising:
a droplet producing apparatus configured to accept cells in a medium and create a plurality of droplets, each droplet comprising a single cell;
an incubator configured to allow replication of cells inside each droplet;
a droplet breaking apparatus adapted to accept the plurality of droplets from the incubator and yield cells in suspension;
a cell culture apparatus adapted to accept cells in suspension from the droplet breaking apparatus, the cell culture apparatus configured to allow for creation of supernatant for testing for cell isolates;
a liquid handling system adapted to create an array of aliquots of supernatant, wherein each aliquot corresponds to each cell isolate; and
a high-throughput mass spectroscopy (HTP-MS) instrument, the high-throughput mass spectroscopy instrument configured to test each aliquot.

11. The system of claim 10, wherein the HTP-MS instrument comprises an ultraviolet (UV) ionization component.

12. The system of claim 10, wherein the HTP-MS instrument is adapted to determine an amount of a desired product or analyte produced by each cell isolate as a function of time.

13. The system of claim 10, wherein the droplet producing apparatus is further configured to accept the cells in suspension created by the droplet breaking apparatus.

14. The system of claim 10, further comprising a cell dilution apparatus adapted to accept the cells in suspension created by the droplet breaking apparatus, wherein the cell dilution apparatus is configured to provide the droplet producing apparatus with cells in the medium.

15. The system of claim 10, wherein the droplets are created using a surfactant in a fluorinated oil, further wherein the droplet breaking apparatus comprises a liquid capable of extracting and repartitioning the surfactant.

16. The system of claim 10, wherein the system is configured to repeat, until a predetermined number of repetitions is achieved, steps comprising:
incubating the cells for a predetermined time period;
accepting, by a cell dilution apparatus, cells in suspension from the droplet breaking apparatus; and
providing, to the droplet producing apparatus, cells in the medium from the cell dilution apparatus.

17. The system of claim 16, wherein the predetermined time period is at least 12 hours.

18. The system of claim 16, wherein the predetermined number of repetitions is at least 2 times.

19. The system of claim 16, wherein the steps further comprise creating cultures in a multi-well plate from the cells in suspension received from the droplet breaking apparatus.

20. A method of determining an amount of a product produced by candidate cells in culture using the system of claim 10, the method comprising:
performing a dALE process, the process comprising:
creating, using the droplet producing apparatus, a plurality of droplets from an initial suspension of cells;
incubating the plurality of droplets in a select environment using the incubator to yield an incubated plurality of droplets;
providing the incubated plurality of droplets to the droplet breaking apparatus;
breaking the plurality of droplets using the droplet breaking apparatus to yield cells in suspension; and
re-encapsulating, by the droplet producing apparatus, the cells in suspension provided by the droplet breaking apparatus; and
after multiple iterations performing the dALE process, measuring the amount of the product or analyte produced by the cells and a rate of replication of the cells.
